(19) United States
(12) Patent Application Publication
(10) Pub. No.: US 2017/0143543 A1
(43) Pub. Date: May 25, 2017

(54) PROCESSES AND APPARATUS FOR PREVENTING, DELAYING OR AMELIORATING ONE OR MORE SYMPTOMS OF PRESBYOPIA

(71) Applicant: Lenticular Research Group, LLC, Naperville, IL (US)

(72) Inventor: George J. McArdle, Naperville, IL (US)

(21) Appl. No.: 15/357,859

(22) Filed: Nov. 21, 2016

Related U.S. Application Data
(60) Provisional application No. 62/258,351, filed on Nov. 20, 2015.

(51) Int. Cl. A61F 9/008

(52) U.S. Cl. CPC A61F 9/00808; A61F 2009/0087; A61F 2009/00895

(57) ABSTRACT

The present invention generally relates to an apparatus and processes for preventing, delaying or ameliorating presbyopia. More particularly, the present invention relates to processes and apparatus for ablating cells in the transitional zone of the crystalline lens of the eye so that onset or progression of presbyopia or one or more symptoms is delayed or prevented.

### CROSS-REFERENCE TO RELATED APPLICATION

This application claims the benefit of priority from U.S. Provisional Application No. 62/258,351, filed on Nov. 20, 2015, the entire content of which is incorporated herein by reference in its entirety.

### FIELD OF THE INVENTION

The present invention generally relates to processes and apparatus for preventing, delaying or ameliorating presbyopia. More particularly, the present invention relates to processes and apparatus for ablating transitional zone cells in the crystalline lens of the eye so as to maintain, increase or restore accommodative ability of the crystalline lens.

### BACKGROUND OF THE INVENTION

Presbyopia is the impairment of vision due to advancing years or old age. Presbyopia is what causes a middle-aged or older person to hold a newspaper, magazine, or book at arm's length to read it. Many presbyopic individuals wear bifocals to help them cope with presbyopia. Presbyopia is typically observed in individuals over 40 years of age. Individuals suffering from presbyopia may have normal vision, but the ability to focus on near objects is at least partially lost over time, and those individuals come to need glasses for tasks requiring near vision, such as reading. Presbyopia affects almost all individuals over the age of 40 to a greater or lesser degree.

For an eye to produce a clear image of objects at different distances, the effective focal length of the eye is adjusted to keep the image of the object focused on the retina at the back of the eye. Accommodation refers to this change in effective focal length. Accommodation is the ability of the eye to change its focus and is accomplished primarily by varying the shape of the crystalline lens. Accommodation provides the ability to change focus from distant objects to near objects. The ability to change focus from distant objects to near objects is impacted by presbyopia.

The shape of the crystalline lens is varied by the use of certain muscles and structures within the eye. As shown in FIG. 1, the crystalline lens 114 is located in the forward part of the eye. The crystalline lens has a generally circular cross-section having two convex refracting surfaces. The curvature of the posterior surface of the lens (which is nearer to the vitreous body) is greater than that of the anterior surface. The crystalline lens is suspended by a circular assembly of collagenous fibers called zonules 104, which are attached at their inner ends to the lens capsule (the outer surface of the crystalline lens) and at their outer ends to the ciliary body 115, a muscular ring of tissue located just within the outer supporting structure of the eye, the sclera 101. As the ciliary muscle contracts the diameter of the ciliary body is reduced, which reduces zonular tension on the crystalline lens. As a consequence, the anterior and posterior curvatures of the crystalline lens increase during accommodation, while the diameter of the lens decreases. When the eye focuses on distant objects, the ciliary muscle relaxes causing the diameter of the ciliary body to increase, which increases zonular tension on the lens. In the unaccommodated state, the anterior and posterior curvatures of the crystalline lens decrease while the diameter increases, and the crystalline lens becomes flattened.

During an individual's life, the crystalline lens continues to grow by epithelial cell division at the equator of the crystalline lens, movement of differentiated cells to a transitional zone, and formation of differentiated fiber cells from some epithelial cells which have moved to the transitional zone. Presbyopia is generally believed to occur at least partially because of continued growth of the crystalline lens. One result of such growth is a progressive reduction in the flexibility of the crystalline lens, thus leading to the continuous decrease of accommodation.

Previous approaches to treating presbyopia have been addressed to the cornea or sclera of the eye, although there have been suggestions to treat presbyopia by addressing the crystalline lens. One technique, called photophako reduction (PPR), would employ a laser to create cavities in the lens, thereby reducing its size. Another technique, called photophako modulation (PPM) would employ a laser to create minute perforations in the lens to soften it. Another technique involves using a photodisruptive laser to soften the inside of the crystalline lens to restore elasticity.

Other attempts to treat presbyopia have involved addressing the sclera or zonules. Laser Presbyopia Reversal (LAPR) involves using infrared lasers for ablation of parts of the sclera. Surgeons use the lasers to make spoke-like excisions in the sclera to thin it and give the lens more room to function. Another approach called Anteríor Ciliary Sclerotomy (ACS) has attempted to make the fibers attached to the lens taut by placing several partial thickness incisions on the sclera or white part of the eye in a radial pattern. Some surgeons have placed silicone implants inside the radial incisions, trying to prevent the regression. Yet another technique for tightening the lens fibers involves applying an infrared laser to strategically thin the sclera.

U.S. Pat. No. 5,465,737 (Schachar) and other patents issued to the same inventor describe treating presbyopia and hyperopia by a method which increases the amplitude of accommodation by increasing the effective working distance of the ciliary muscle in the presbyopic eye. Schachar states that presbyopia is also arrested by inhibiting the continued growth of the crystalline lens by application of heat, radiation or antimitotic drugs to the epithelium of the lens.

Other techniques for treating presbyopia have been suggested, the most common being addressed to the cornea or the sclera. See, for example, U.S. Pat. No. 6,258,082; U.S. Pat. No. 6,263,879; U.S. Pat. No. 6,491,688; U.S. Pat. No. 6,663,619; U.S. Pat. No. 6,745,775; U.S. Pat. No. 5,312,320; U.S. Pat. No. 5,711,762; U.S. Pat. No. 5,735,843; U.S. Pat. No. 6,325,792; U.S. Pat. No. 6,706,036; and U.S. Pat. No. 5,439,462. U.S. Pat. No. 7,252,662 (McArdle et al.) generally relates to an apparatus and processes for preventing or delaying presbyopia. More particularly, it relates to processes and apparatus for ablating epithelial cells in the germinative zone or the pregerminative zone of the crystalline lens of the eye so that onset or progression of presbyopia or one or more symptoms is delayed or prevented.

U.S. Pat. No. 8,991,401 (McArdle et al.) generally relates to an apparatus and processes for preventing or delaying presbyopia. More particularly, it relates to processes and apparatus for ablating epithelial cells in the germinative zone or the pregerminative zone of the crystalline lens of the eye so that onset or progression of presbyopia or one or more symptoms is delayed or prevented. It also relates to processes and apparatus for promoting formation of suture lines in the crystalline lens of the eye so that onset or progression of presbyopia or one or more symptoms is delayed or prevented. It also relates to processes and apparatus for creating disruptions in the vitreous humor of the eye.

### SUMMARY OF THE INVENTION

As one aspect of the present invention, a process is provided for preventing or delaying presbyopia in a subject, wherein the subject has a crystalline lens having a transitional zone, the process comprising the step of ablating cells in a transitional zone of a crystalline lens. The process can include making one or more ablation points are made in the transitional zone of the crystalline lens. As discussed below, a crystalline lens includes a germinative zone, a pregerminative zone, and fiber cells in addition to the transitional zone. In some embodiments, the process comprises distinguishing between cells in the transitional zone and cells in the germinative and pregerminative zones, and the process may also comprise excluding the ablating of cells in the germinative or pregerminative zones of the crystalline lens. In some embodiments, the process comprises distinguishing between cells in the transitional zone and fiber cells, and the process may comprise excluding the ablation of fiber cells.

As another aspect of the present invention, a process is provided for preventing or delaying one or more symptoms of presbyopia in a subject. The process comprises the steps of: selecting a subject prior to detecting a symptom of presbyopia in the subject, and ablating cells in the transitional zone of the crystalline lens. In some embodiments, the subject is selected based on an increased risk factor for presbyopia and/or based on age.

### BRIEF DESCRIPTION OF THE FIGURES

FIG. 1 shows the interior of a human eye, including the crystalline lens.

FIG. 2 shows the arrangement of epithelial cells in a crystalline lens of a human.

FIG. 3 shows the various zones of epithelial cells in a crystalline lens.

### DETAILED DESCRIPTION

Processes and apparatus are provided for preventing or delaying the onset or progression of presbyopia, and for ameliorating presbyopia or one or more symptoms of presbyopia. The onset and progression of presbyopia are typically manifested through one or more symptoms of presbyopia, and the present apparatus and processes can be used to prevent or delay one or more symptoms of presbyopia. The apparatus and processes can be used to prevent or delay the onset of presbyopia before a subject is diagnosed with or begins to suffer from one or more symptoms of presbyopia. The apparatus and process can be used to prevent or delay the progression of presbyopia so that one or more symptoms of presbyopia does not become worse or more noticeable. The present treatment processes include therapeutic treatments (to ameliorate one or more symptoms of presbyopia) and preventative treatments (to prevent or delay the onset or progression of presbyopia).

The present processes and apparatus can be used to ameliorate presbyopia or one or more symptoms of presbyopia. As used in this disclosure, presbyopia is ameliorated if one or more of its symptoms is reduced, attenuated, diminished, mitigated, cured or eliminated, either temporarily or permanently. Amelioration can be detected using diagnostic techniques or by self-reporting from a human subject.

Symptoms of presbyopia include decreased focusing ability for near objects, eyestrain, difficulty reading fine print, fatigue while reading or looking at an illuminated screen, difficulty seeing clearly up close, less contrast when reading print, need for brighter and more direct light for reading, needing to hold reading material further away in order to see it clearly, and headaches, especially headaches when using near vision.

In at least one embodiment, the present methods are employed with a crystalline lens of a subject, such as invertebrate or vertebrate animals. In at least one embodiment, the subject is an insect, arachnid, microorganism, reptile, mammal, amphibian, bird or fish. In some embodiments, the subject is human. In some embodiments, the subject is selected from the group consisting of humans, primates, dogs, pigs, rabbit, rats, mice, and guinea pigs. In some embodiments, the subject is selected from the group consisting of humans, primates, dogs, cats, rats, mice, horses, cows, pigs, fish, birds, sheep, goats, frogs, rabbits, or guinea pigs.

The processes and apparatus address the onset or progression of presbyopia by ablating transitional zone cells in the crystalline lens.

In some embodiments of the present processes, transitional zone cells are ablated, using a laser, microwave energy, RF energy, ultrasound, or other ablative technique. More particularly, some cells in the transitional zones are ablated so as to slow or prevent growth or compaction of the crystalline lens. Ablating cells means removing cells, including by cutting, extirpating, vaporizing, abrading, or any other suitable technique for removing cells from a living tissue. By ablating cells in the transitional zone of the crystalline lens, it is contemplated that elimination of a transitional zone cell will prevent formation of a fiber cell. When using a laser-based surgical technique, ablated cells are usually vaporized. Cells in the transitional zone of the crystalline lens can be ablated by any suitable technique, but will generally be ablated using laser-based surgical techniques. Other details regarding laser based surgical techniques, including various laser sources, as well as other ablative techniques are discussed below.

Treatment processes will generally include the step of dilating the pupil in order to expose more of the crystalline lens. Dilation will facilitate exposure and treatment of the peripheral portions of the crystalline lens, including the germinative zone. Dilation is useful because the present techniques are to be applied to the lens rather than the iris, and the iris normally is disposed above the area of transitional zone. In the present methods, the iris is dilated beyond what is usual for other procedures, to an extent where the transitional zone is no longer covered by the iris. Such dilation may be done by using higher concentrations of dilating agents than usual. For example, the methods can comprise administering a dilating agent selected from atropine at greater than 1%, for example 2% or more, or 3% or more; cyclopentolate at a concentration greater than 2%, for example 2.5% or more, or 3% or more; homatropine at a concentration greater than 5%, for example 7.5% or more, or 10% or more; or tropicamide at greater than 1%, for example 2% or more, or 3% or more.

Treatment processes can also include the step of visually identifying the cells in the transitional zone of the crystalline lens. The cells can be identified when viewed microscopically as distinct from epithelial cells and from fiber cells, such as by their size, shape, lack of nuclei, and other morphological features. In some embodiments, a label is topically administered to the eyes of the subjects. This label, or a moiety linked to the label, specifically targets cells, nuclei or other organelles exclusively or predominantly found in cells in the transitional zone, so that cells in the transitional zone can be distinguished from cells in the germinative zone and/or from fibers cells.

Treatment processes can also include one or more of the steps of determining the growth rate and/or compaction rate of a crystalline lens or production rate of fiber cells in the crystalline lens, and estimating the amount of transitional zone cells to be ablated for stopping or slow growth or compaction. If those rates are determined, an approximation can be made regarding the extent to which the cells of the transitional zone should be ablated in order to achieve the desired decrease in fiber cell formation.

Treatment processes can include one or more of the steps of determining the suture pattern of an eye before treatment and determining the suture pattern after treatment.

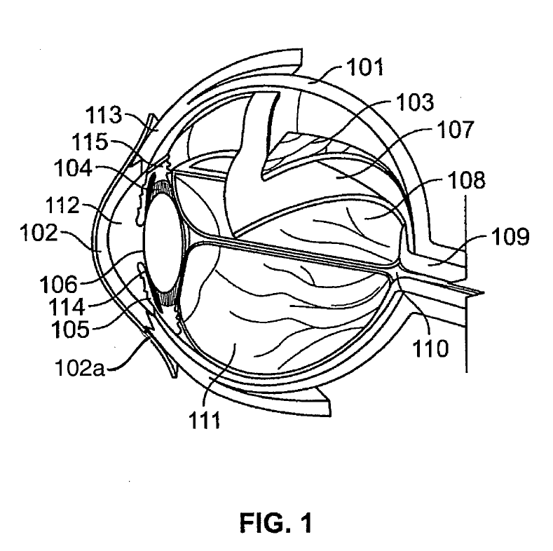

FIG. 1 shows various structures of the human eye. The outermost layer of the eye is called the sclera 101, which is commonly referred to as “the white of the eye.” The sclera 101 is the tough, opaque tissue that serves as the eye's protective outer coat. Tiny muscles connect to the sclera 101 around the eye and control the eye's movements. The sclera 101 maintains the shape of the eye.

The cornea 102 is at the front of the eye. Light passes through the cornea 102 when it enters the eye. The transparent cornea 102 and the opaque sclera 101 meet at the corneal-scleral junction, generally indicated at 102a in FIG. 1. The cornea is arranged in layers, namely epithelium, Bowman's layer, the stroma, Descemet's Membrane, and the endothelium. The epithelium is the cornea's outermost region. The epithelium blocks the passage of foreign material, provides a smooth surface that absorbs oxygen and cell nutrients from tears, then distributes these nutrients to the rest of the cornea. The epithelium is filled with thousands of tiny nerve endings that make the cornea extremely sensitive to pain when rubbed or scratched. The present apparatus and processes are designed to avoid damaging or inflicting pain on the cornea (including the epithelium layer). Under the epithelium is a transparent sheet of tissue called Bowman's layer. Bowman's layer is composed of strong layered protein fibers called collagen. If injured, Bowman's layer can form a scar as it heals. If these scars are large and centrally located, some vision loss can occur. Accordingly, the present processes and apparatus are designed to avoid damage to Bowman's layer or other layers containing collagen. Under Bowman's layer is the stroma, which provides most of the cornea's thickness. It is mostly water and collagen. Collagen gives the cornea its strength, elasticity, and form. The collagen's shape, arrangement, and spacing produce the cornea's light-conducting transparency. Under the stroma is Descemet's membrane, a thin but strong sheet of tissue that serves as a protective barrier against infection and injuries. Descemet's membrane includes collagen fibers (different from those of the stroma) and is made by the endothelial cells that lie below it. The endothelium pumps excess fluid out of the stroma. If endothelium cells are damaged by disease or trauma, they are not repaired or replicated. If too many endothelial cells are destroyed, corneal edema and/or blindness may ensue. Once again, the present processes and apparatus are designed to avoid damaging the layers of the cornea (including the endothelial cells of the cornea) when used to treat a subject for the prevention of presbyopia.

Returning to FIG. 1, the choroid 103 (or uveal tract) contains the blood vessels that supply blood to structures of the eye. The front part of the choroid 103 contains: ciliary body 115 which is a muscular area and the zonules 104 that are attached to the lens 114. The ciliary body 115 contracts and relaxes to control the zonules 104, which in turn control the size of the crystalline lens for focusing. The iris 105 is the colored part of the eye. The color of the iris is determined by the color of the connective tissue and pigment cells. Less pigment makes the eyes blue; more pigment makes the eyes brown. The iris is an adjustable diaphragm around an opening called the pupil 106. The iris 105 may be moved by dilating the pupils by administration of eye drops, for example, mydriatics, such as atropine, cyclopentolate, homatropine, phenylephrine, scopolamine, and tropicamide. Ophthalmologists routinely dilate subjects' eyes as part of eye exams.

The retina 107 is located at the back of the eye. The retina 107 is the light-sensing portion of the eye. The macula 108 is in the center of the retina, and in the center of the macula is an area called the fovea centralis. This area is responsible for seeing fine detail clearly. Retinal nerve fibers collect at the back of the eye and form the optic nerve 109, which conducts the electrical impulses to the brain. The optic nerve 109 is connected to the sclera 101 at the back of the eye. The spot where the optic nerve and blood vessels exit the retina is called the optic disk 110. This area is a blind spot on the retina because there are no rods or cones at that location.

The eye has two fluid-filled sections separated by the crystalline lens 114. The larger, back section contains a clear, gel-like material called vitreous humor 111. The smaller, front section contains a clear, watery material called aqueous humor 112. The aqueous humor is divided into two sections called the anterior chamber (in front of the iris) and the posterior chamber (behind the iris). The aqueous humor is produced in the ciliary body 115 and is drained through the canal of Schlemm 113. If this drainage is blocked, glaucoma can result.

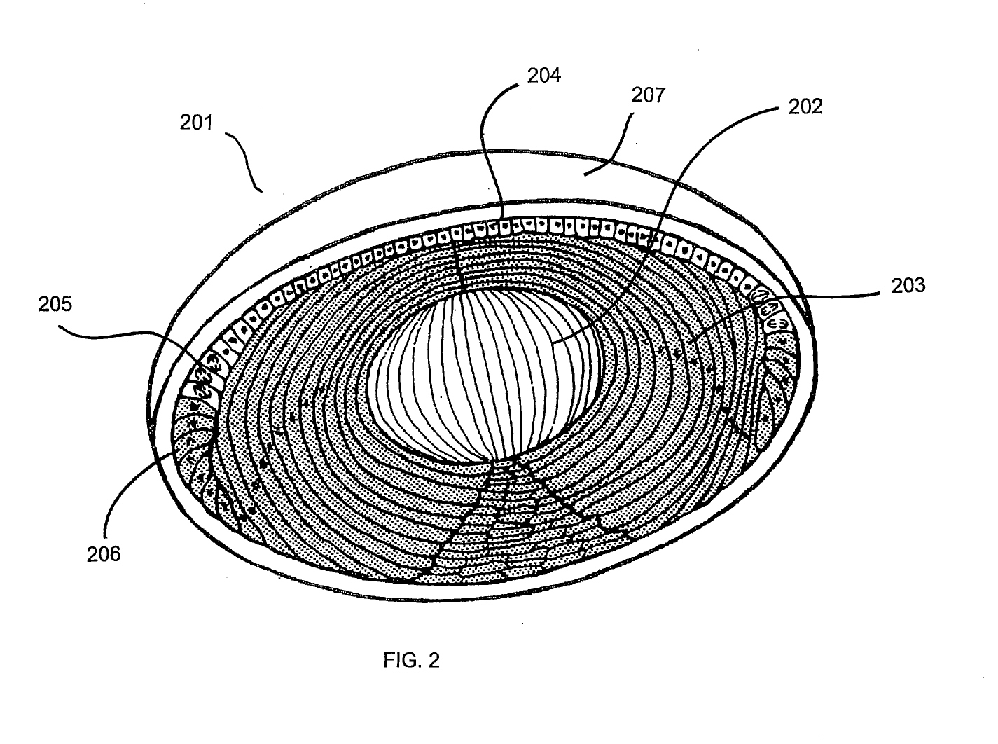

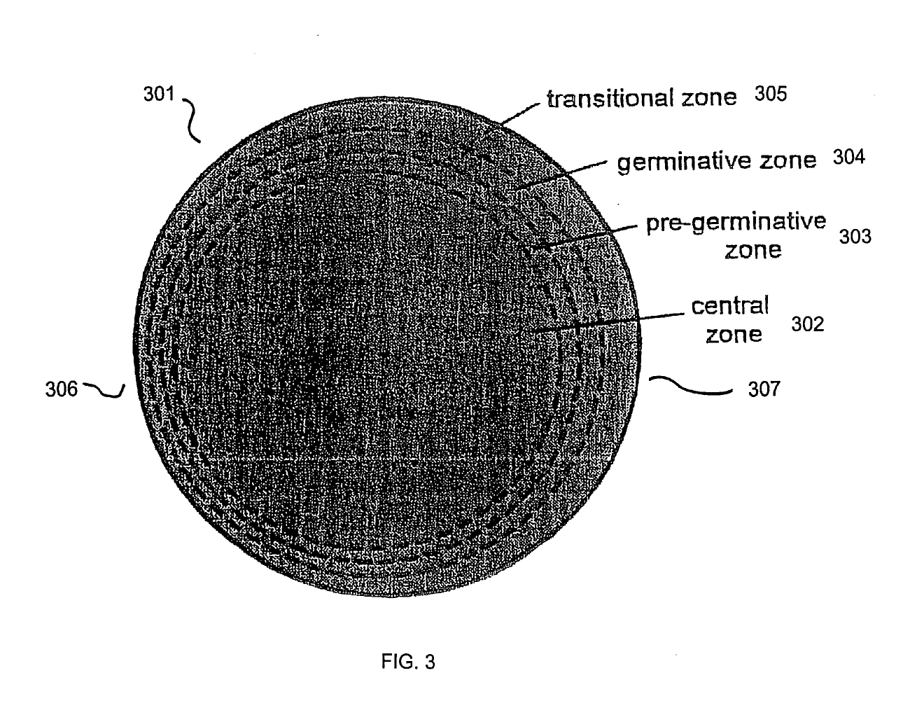

The crystalline lens 114 is a clear, biconvex structure about 10 mm (0.4 inches) in diameter in an average adult and smaller in children. The crystalline lens changes shape because it is attached to muscles in the ciliary body. The crystalline lens 114 is used for dynamic focusing. Additional details about the crystalline lens are provided in FIGS. 2 and 3 and the descriptions below, as well as in Kuszak et al., Electron Microscopic Observations of the Crystalline Lens, Microscopy Research and Technique 33:441-79 (1996) and Kuszak et al., Biology of the Lens: Lens Transparency as a Function of Embryology, Anatomy, and Physiology, In: The Principles and Practice of Ophthalmology (2nd ed.), edited by Albert D A and Jacobiec F A. Philadelphia, Pa.: Saunders, 1999, p. 1355-1408, both of which are incorporated by reference herein.

The present processes and apparatus involve the anatomy of the crystalline lens. The adult human crystalline lens is an asymmetric, oblate spheroid. The crystalline lens is an intricate arrangement of highly specialized cells that produce a gradient of refractive index.

FIG. 2 shows the general structure of a crystalline lens 201. The crystalline lens is a transparent, biconvex structure with an anterior half that is less spherical, than the posterior half. The core of the crystalline lens comprises a nucleus of primary lens fibers 202 which are elongated along the visual axis. The core is surrounded by a cortex of elongated secondary lens fibers 203. At the anterior face of the lens resides a layer of cuboidal cells 204 which make up the central zone of the lens 201. An anterior monolayer 205 serves as the germ cell layer of the lens, a stratified epithelia-like tissue. However, unlike other stratified epthelia that have their stem cells distributed throughout a basal germ cell layer, stem cells of the lens are sequestered as a narrow latitudinal band within the lens epithelium, forming the germinative zone of the crystalline lens. The germinative zone lies at the periphery of the lens epithelium just above the lens equator. Some of the germinative zone cells undergo mitotic division, and a number of the daughter cells terminally differentiate to become additional lens fibers. Differentiating cells in the process of becoming lens fibers 206 are found outside their germinative zone in the transitional zone. Because these are the second lens fibers to develop, they are referred to as secondary fibers 203. The epithelial cells of the crystalline lens are covered by a noncellular outer covering called the capsule 207.

FIG. 3 generally shows that the lens epithelial cells tend to be sequestered in distinct zones within the lens epithelium. A central zone 302 comprises a broad polar cap of lens epithelium that covers most of the anterior surface of the lens. Central zone cells are held in the GO stage of the cell cycle and, therefore, do not contribute to secondary fiber formation. Between the central zone 302 and the germinative zone 304 is a relatively narrow zone called the pregerminative zone 303. A small number of pregerminative zone cells undergo mitosis, and some of these daughter cells terminally differentiate into secondary lens fibers. Finally, beyond the germinative zone is a narrow latitudinal band of cells called the transitional zone 305.

Transitional zone cells are the cells that have undergone mitosis in the germinative zone and have been selected to terminally differentiate into secondary lens fibers. However transitional zone cells still have nuclei and organelles, at least in part, which distinguishes them from fiber cells. As additional germinative zone cells are recruited throughout life to become secondary lens fibers, the transitional zone cells are forced to migrate posteriorly. During the migration of these nascent secondary lens fibers, they simultaneously rotate 180 degrees about their polar axis, and then elongate bidirectionally until they become mature secondary lens fibers. As elongation proceeds, the anterior ends of the initial elongating secondary lens fibers are insinuated beneath the apical membranes of the overlying lens epithelium and above the anterior ends of the primary lens fibers. Simultaneously, the posterior ends of the same elongating secondary lens fibers are insinuated beneath the lens capsule and above the posterior ends of the primary lens fibers. Secondary lens fiber elongation is complete, and fibers are considered mature when they are arranged end to end as a complete growth shell, rather than as a layer or stratum, as is typical of most stratified epthelia.

As additional secondary lens fibers develop throughout life, their anterior ends are insinuated beneath the apical membranes of the lens epithelium and above the anterior ends of previously formed lens fibers, while their posterior ends are insinuated above the capsule and beneath the basal membranes of the same previously formed lens fibers. The ends of the lens fibers meet to form a line called a suture line or suture. In this manner, lens fibers of every shell lie atop lens fibers of the previously formed shell and beneath the lens fibers of the subsequently formed shell. In addition, the entire lens mass is enclosed in a basement membrane-like capsule, that is produced by the basal membrane of the lens epithelial cells and elongating lens fibers. As a result of its continuous production throughout life, the lens capsule becomes the thickest basement membrane in the body.

Sutures are formed as an end point of secondary fiber development. The anterior ends of defined pairs of fibers abut and overlap with one another, as do their posterior ends, to form “sutures” thereby completing a growth shell. Suture patterns are not identical in different species. There are four distinct types of lens sutures. In order of increasing complexity the suture types are the umbilical, line, Y, and star. Many animal models used in lens research have relatively simple suture types. Chickens have lenses with umbilical sutures while frogs and rabbits have line sutures. Mice, rats, and other animals have lenses with Y sutures. Primate lenses have Y sutures throughout gestation, but then develop and grow progressively more complex iterations of star sutures. Additional discussion and details about suture formation and suture patterns are found in Kuszak et al., “Development of Lens Sutures,” Int. J. Dev. Biol. 48: 889-902 (2004); Kuszak et al., “Computer modeling of secondary fiber development and growth: 1. Nonprimate lenses,” Molecular Vision 2006; 12:251-270; and Kuszak et al., “Fibre cell organization in crystalline lenses”, Experimental Eye Research, 78:3, 673-687 (2004).

Unlike other stratified epithelia, the crystalline lens does not routinely slough off cells from its older, uppermost strata. Instead, the older lens cells are progressively more internalized throughout life. In this manner, the crystalline lens retains all of its lens fibers arranged in order of ascending age from its periphery to its interior.

At any age, the germinative zone 303 comprises approximately the outer 10% of the anterior surface of the lens epithelium (additionally, the transitional zone comprises the most peripheral segment of this area). The central zone 302 and pregerminative zone 304 account for the remaining 90% of the anterior surface area of the lens epithelium. Although all zones of the lens epithelium increase in size as a function of age, mitotic activity is restricted primarily to the germinative zone.

As mentioned above, lens epithelial cells are separated into distinct subpopulations. Adult lens central zone epithelial cells are cuboidal 204 with an average height of 3 to 7 μm. Pregerminative zone cells and germinative zone cells are generally smaller. The germinative zone may be identified on the crystalline lens by reference to latitudinal and longitudinal coordinates, for example, from 90 degrees (the top of the crystalline lens) to about 75 to about 80 degrees latitude is the central zone. The germinative zone is from about 0 degrees to about 10 degrees latitude. The longitudinal coordinates can be between 0 and 90 degrees, though preferably symmetrical longitudinal coordinates are employed.

The proliferation of fiber cells in the crystalline lens can be reduced by preventing, slowing or stopping transitional zone cells from fully becoming fiber cells, such as by ablating such cells. If the generation of a significant percentage of fiber cells is prevented, slowed or stopped, growth of the crystalline lens can be brought to or near a stasis. Moreover, by inhibiting the creating of new fiber cells, the compaction of cells within the crystalline may be reduced or slowed. These effects can prevent or delay the onset or progression of one or more symptoms of presbyopia.

Ablation of transitional zone cells according to the present techniques does not require a measurable or immediate decrease the equatorial diameter of the crystalline lens. Transitional zone cells are ablated so as to stop or slow growth of the crystalline lens. In some embodiments, the process for preventing, delaying or ameliorating the symptoms of presbyopia can include ablating transitional zone cells in the crystalline lens without stopping growth of the crystalline lens; that is, the crystalline lens may continue to experience some growth and become somewhat larger or more compacted, but the rate of growth is slowed, and as a consequence, symptoms of presbyopia are prevented, delayed or ameliorated.

The present processes are primarily designed to symmetrically ablate transitional zone cells. When an ablative point is made on the crystalline lens, preferably there is also one or more additional ablation points made to form a symmetric pattern with the first (and any other) ablation point, such that the ablation points are symmetrically disposed around the crystalline lens.

While some types of laser surgery may employ either an even or odd number of ablation points (for example, providing holes in the iris for relieving pressure for glaucoma), it is contemplated that an even number of ablation points can be preferable for the present processes. Where a number of ablation points are made in the crystalline lens, it is desirable that the ablation pattern is symmetrical and that the number of degrees between each ablative point is approximately the same.

It is desirable in the present processes to provide symmetrical ablation or another symmetrical mechanical or other approach. For example, it is generally desirable to provide symmetrical ablation of transitional zone cells in the crystalline lens. This is because it is highly desirable, when dealing with the crystalline lens to promote symmetrical movement of cells and fiber growth and symmetrical formation of sutures, so as to avoid or reduce disruption of visual clarity.

Accordingly, the present processes will preferably yield an even number of ablation points or openings (although an odd number may be suitable in some circumstances). More preferably the present processes yield a symmetric pattern of an even number of ablation points around essentially the entire circumference of the crystalline lens, or a symmetric pattern of an even number of openings around essentially the entire circumference of the corneal-scleral junction. For example, if six ablation points were to be made in the crystalline lens or the corneal-scleral junction at various degrees longitude, ablation points could be made at 90 degrees, 91 degrees, 92 degrees, and at 270 degrees, 271 degrees, and 272 degrees, because that would result in having three ablation points on either side, though the regions at zero degrees and 180 degrees would not be ablated. However, it would be more desirable to have the ablation points at 0 degrees, 60 degrees, 120 degrees, 180 degrees, 240 degrees, and 300 degrees, so that the ablation points were symmetrical around the entire circumference of the crystalline lens or the corneal-scleral junction.

Preferably, the transitional zone cells are ablated symmetrically around the crystalline lens or the corneal-scleral junction and/or an even number of ablation points are made. For example, at least 4 symmetrical ablation points are made, such as at about 0 degree longitude, about 90 degrees longitude, about 180 degrees longitude, and about 270 degrees longitude around the circumference of the germinative zone or the pregerminative zone. As another example, at least 12 symmetrical ablation points are made, such as at about 0 degree, about 30 degrees, about 60 degrees, about 90 degrees, about 120 degrees, about 150 degrees, about 180 degrees, about 210 degrees, about 240 degrees, about 270 degrees, about 300 degrees, and about 330 degrees (all in degrees longitude), around the circumference of the germinative zone or the pregerminative zone of the crystalline lens or around the circumference of the corneal-scleral junction.

By using symmetric ablation, it is believed the risk of causing a cataract or an astigmatic condition is reduced. A cataract is a clouding of the crystalline lens. An astigmatic condition would occur where the crystalline lens distorts because the progression of cell growth and/or multiplication around the circumference of the crystalline lens has not been uniformly slowed or stopped. By using symmetric ablation, it is believed that the likelihood of maintaining optical clarity of the crystalline lens is improved.

It is desirable to make ablation spots in a manner which maintains optical clarity of the crystalline lens. To that end, it is desirable to maintain fiber cell growth toward existing or new suture lines in the crystalline lens in a symmetrical or geometric pattern and to avoid growth of the fiber cells in a different or haphazard fashion. Suture lines are the end-to-end junctions of the fiber cells which are aligned with each other.

Ablating transitional zone cells in a symmetrical pattern reduces the risk that epithelial cells will repair perceived damage from ablation in a manner which interferes with optical clarity. Fibers in the process of differentiating rely on epithelial cells for critical information. Moreover, ablating some transitional zone cells can decrease the provision of differentiation support factors, thereby reducing the reproduction and/or differentiation of unablated transitional zone cells.

In some embodiments of the present processes, a sufficient number or arrangement of ablation points or openings or disruptions are made so as to provide new epithelial cells and/or new fiber cells. The present processes may comprise making a number or arrangement of ablation points or openings to prevent or delay the onset or progress of presbyopia or one or more symptoms, or to ameliorate presbyopia or one or more symptoms. A suitable number of ablation points or openings may be, for example 2, 3, 4, 6, 8, 12, 16, 20, 24, 28, 30, 60, 120, 180, 360, 480, 540, 600, 660, 720, 800, 840, or 960 ablation points. Furthermore it may desirable to make even larger numbers of ablation points or openings or disruptions, for example about 1000, 1800, 2000, 2400, 3000, 3600, 4000, 4800, 5000, 6000, 7000, 7200, 8000, 8800, 9000, 9600 or 10000. Any two of the foregoing numbers may be combined to form a range of ablation points or openings.

Preferably, the present mechanical, chemical or other techniques are employed so that growth of the crystalline lens is promoted. For example, openings are made in the corneal-scleral junction, or disruptions are created in the vitreous humor, or epithelial cells in the germinative or pregerminative zone are sufficiently ablated, so as to establish a growth rate that is about 100% or more of the pre-ablative growth rate, alternatively about 110% or more. Alternatively, a growth rate is established that is at least about 100.1%, 100.2%, 100.3%, 100.5%, 101%, 102%, 103%, 104%, 105%, 110%, 115%, 120%, 125%, 130%, 140%, 150%, 175%, or 200%, of the pre-treatment growth rate. This increased growth rate is not expected to be permanent, but rather may last for a minimum or maximum period of time, such as one week, two weeks, one month, two months, three months, one year, or longer. Any of the foregoing values may be combined to form a range.

The present processes comprise making a sufficient number of ablation points, openings or disruptions without seriously damaging the function or structure crystalline lens, such as by the formation of a significant cataract or astigmatic condition. For example, it is contemplated that not each and every cell in the transitional zone will be ablated, but rather a percentage of such cells. In preferred embodiments, at least about 10% of the cells in the transitional zone of the crystalline lens are ablated. Alternatively, at least about 0.001%, at least about 0.01%, at least about 0.1%, at least about 1%, at least about 2%, at least about 5%, at least about 7%, at least about 10%, at least about 12%, at least about 15%, at least about 18%, at least about 20%, at least about 25%, at least about 30%, at least about 35%, at least about 40%, at least about 50%, at least about 60%, or more of the epithelial cells in the germinative zone and/or the pregerminative zone of the crystalline lens are ablated. Alternatively a percentage of the circumference of the germinative or pregerminative zone may be ablated. For example, from about 0.001% up to 100% of the circumference of the crystalline lens can be ablated.

Preferably, cells in the transitional zone of the crystalline lens are sufficiently ablated so that the lens regains or retains accommodative capacity. Alternatively, cells in the transitional zone are sufficiently ablated to establish a reduced density that is about 95% or less of the pre-ablative density, alternatively about 90% or less. Alternatively, cells in the transitional zone are sufficiently ablated to establish a density that is at most about 85%, 80%, 75%, 70%, 65%, 60%, 55%, 50%, 45%, 40%, 35%, 30%, 25%, 20%, 15%, 10%, 5%, 3%, 2%, or 1% of the pre-ablative density. It is presently preferred to reduce the density by the minimum amount that restores the desired flexibility of the crystalline lens.

The present processes comprise ablating a sufficient number of cells in the transitional zone without ablating so many cells that the function or structure crystalline lens is seriously damaged, such as by the formation of a significant cataract or astigmatic condition. It is contemplated that not each and every cell in the transitional zone will be ablated, but rather a percentage of such cells. In preferred embodiments, at least about 1% of the cells in the transitional zone of the crystalline lens are ablated. Alternatively, at least about 0.001%, at least about 0.01%, at least about 0.1%, at least about 1%, at least about 2%, at least about 5%, at least about 7%, at least about 10%, at least about 12%, at least about 15%, at least about 18%, at least about 20%, at least about 25%, at least about 30%, at least about 35%, at least about 40%, at least about 50%, at least about 60%, or more of the cells in the transitional zone of the crystalline lens are ablated.

The number of ablation points or ablation lines made in the present processes will depend in part on the size and shape of the ablation points and/or the dimensions of the ablation line. For example, fewer ablation points or ablation lines will usually be made when the ablation points or ablation lines are larger. Sizes for the ablation points include, but are not limited to, ablation points having diameters in the range from about 1.6 microns to about 3000 microns, alternatively from about 3 microns to about 1000 microns, alternatively from about 3.12 microns to about 106 microns. Preferably, ablations points have diameters sizes in the range of from about 3 to about 300 microns. More preferably, the ablation points have diameters which are approximately the same as the width of a cell in the transitional zone, or a whole number multiple of that width. Alternatively, the ablation points may have a diameter that is between about 5 microns and about 10 microns, alternatively between about 6 microns and about 8 microns. Sizes for ablation points further include, but are not limited to, ablation points having volumes in the range of from about 14 cubic microns to about 1.4×10⁷ cubic microns, alternatively from about 140 cubic microns to about 1.4×10⁶ cubic microns, alternatively from about 1400 cubic microns to about 1.4×10⁵ cubic microns. The ablation points can have a shape that is round, square, polygonal, or another shape. For example, the ablation points may have the shape of a circle, curved line or crescent, which may facilitate ablation of a larger number of cells at each ablation point. Similarly, the ablation line may have the shape of a straight line, curved line or crescent. Preferably the ablation line has a shorter dimension that is about the same as the width of a cell, as a whole number multiple of that width. Alternatively, the ablation lines may have a shorter dimension that is between about 5 microns and about 10 microns, alternatively between about 6 microns and about 8 microns. The ablation points can be connected to form a larger and/or different shape or pattern. For example, the ablation points can be connected to form a line, a ring, or circle that is essentially the entire circumference of the crystalline lens.

The processes may further comprise the step of selecting a subject(s) prior to the onset of one or more symptoms of presbyopia, and ablating a number of cells in the transitional zone of the crystalline lens of the selected subject(s). A number of cells are ablated to prevent or delay one or more (preferably all) symptoms of presbyopia. The subject may be selected based on age or based on one or more risk factors for symptoms of presbyopia. For example, the subject may be at least 12 years of age, alternatively at least 15 years of age, alternatively at least 18 years of age, alternatively at least 21 years of age, alternatively at least 25 years of age, alternatively at least 30 years of age, alternatively at least 35 years of age, alternatively at least 40 years of age, alternatively at least 45 years of age, alternatively at least 50 years of age, alternatively at least 55 years of age, alternatively at least 60 years of age, alternatively at least 65 years of age, alternatively at least 70 years of age, alternatively at least 75 years of age, alternatively at least 80 years of age. Alternatively, the subject may be less than 12, 15, 18, 21, 25, 30, 35, 40, 45, 50, 55, 60, 65, 70, 75, or 80 years of age. The subject may be selected based on an increased risk factor for symptoms of presbyopia, such as hyperopia (which puts additional demand on the flexibility of the lens), occupation (which is a risk factor if the occupation requires near vision demands), gender (presbyopia often occurs earlier in females, ocular disease or trauma (damage to the lens or ciliary muscles can accelerate progression of presbyopia), systemic disease (diabetes and multiple sclerosis increased risk for presbyopia), drug use (drugs such as alcohol, anti-depressants, and antihistamines can decrease the flexibility of the lens), or atmospheric or geographic factors (higher annual temperatures and greater exposure to ultraviolet radiation put individuals at an increased risk for presbyopia).

The present processes may further comprise the step of selecting a subject(s) after the onset of one or more symptoms of presbyopia, for example, one or more initial or mild symptoms, and ablating a number of cells to ameliorate one or more of the symptoms or to prevent or delay the onset of one or more advanced or severe symptoms. Examples of initial or mild symptoms are holding an object out further to read or view it, or to view it clearly. Examples of advanced or severe symptoms are significant or complete loss of vision or the ability to focus.

Laser apparatus suitable for ablating cells in the transitional zone are described in detail herein and in the references cited herein (which are incorporated by reference). For example, apparatus for performing laser-based ablative techniques are described in McArdle et al., U.S. Pat. No. 7,252,662.

In some embodiments, the present processes and apparatus will include a laser source capable of generating a laser beam. Preferably, the laser source generates short pulses of laser light having a wavelength which will not damage the cornea and which will not generate substantial thermal damage to the eye. In general, the energy level per pulse for the laser is preferably in the range of from about 0.1 microjoules to about 1200 microjoules, preferably from about 1 microjoule to about 120 microjoules, more preferably about 12 microjoules. Numerous commercially available lasers meet these requirements. Using lasers at very short pulse durations with a relatively predictable power level is desired, so if the laser is well calibrated, there should not be significant differences in the amount of energy provided for each ablation point.

In general, the laser source should provide a beam that is of a wavelength that transmits through the cornea. The beam should be of a short pulse width, together with the energy and beam size. Short pulse lengths are desirable to avoid transferring heat or shock to material being lasered, which means that ablation can be performed with virtually no damage to surrounding tissue. In some embodiments, the present apparatus and methods employ a femtosecond or picosecond laser source or apparatus. Femtosecond lasers are capable of generating pulse lengths on the femtosecond scale, for example less than 10 fs, or less than 100 fs, with pulse frequencies presently as high as 10 KHz or higher. Picosecond lasers are generally laser diode based or LED based devices that can be operated at variable, user-adjustable repetition rates. They can cover discrete wavelengths from 245 nm to 1990 nm or can be tunable in the visible-infrared spectrum. Power levels vary between μW and hundreds of mW for amplified versions. Pulse widths can be less than 1 ps, or less than 10 ps, or less than 100 ps. Some picosecond lasers operate in ultraviolet wavelengths, for example, at 355 nm, use Q-switching technology to deliver single-longitudinal mode pulses with pulsewidths in the few hundred picosecond range from modified diodepumped solid-state (DPSS) laser designs with a very short laser resonator.

Examples of femtosecond and picosecond lasers include the Clark CPA-2161, an amplified Ti:Sapphire having a wavelength of 775 nm, less than a 150 femtosecond pulse width, about 3 KHz PRF, with 850 microjoules; the IMRA FCPA (fiber chirped pulse amplification) microjewel D series D-400-HR, which is a Yb:fiber oscillator/amplifier having a wavelength of 1045 nm, less than a 1 picosecond pulse width, about 5 MHz PRF, with 100 nanojoules; Delmar Photonics Inc. Trestles-20, a Titanium Sapphire (Ti:Sapphire) oscillator having a wavelength range of 780 to 840 nm, less than a 20 femtosecond pulse width, about 100 MHz PRF, with 2.5 nanojoules; Lumera Staccato, a Nd:YVO4 having a wavelength of 1064 nm, about 10 picosecond pulse width, about 100 KHz PRF, with 100 microjoules; and Lumera Rapid, a ND:YVO4 having a wavelength of 1064 nm, about 10 picosecond pulse width, and can include one or more amplifiers to achieve approximately 2.5 to 10 watts average power at a PRF of between 25 kHz to 650 kHz and also includes a multi-pulsing capability that can gate two separate 50 MHz pulse trains, and, the IMRA FCPA (fiber chirped pulse amplification) pJewel D series D-400-NC, which is a Yb:fiber oscillator/amplifier having a wavelength of 1045 nm, less than a 100 picosecond pulse width, about 200 KHz PRF, with 4 microjoules. Thus, these and other similar lasers may be used in the present apparatus and methods.

Laser sources include sources of visible wavelength laser light and infrared laser light. For example, a YAG laser is used, such as a Nd:YAG (Neodymium:Yttrium Aluminum Garnet) laser. Ophthamic Nd:YAG lasers for laser capsulotomy after cataract surgery include the 7970 Coherent YAG laser; Ophthalmic Nd:YAG laser YC-1600 available from Nidek Incorporated of Fremont, Calif.; and the Alcon 2500 YAG Laser. Nd:YAG lasers have been used for ophthalmological surgeries such as posterior capsulotomy and peripheral iridotomy. Nd:YAG lasers generate short pulse, low energy, high power, coherent optical radiation. When the laser output is combined with focusing optics, the high irradiance at the target causes tissue disruption via optical breakdown. Different materials can be included in the YAG crystals that emit very specific wavelengths. In medical applications, homium and thulium are impurities frequently used in the YAG crystal, but they have slightly different wavelengths.

Other laser sources include helium-cadmium lasers, argon ion lasers, krypton ion lasers, xenon lasers, iodine lasers, holmium doped yttrium-aluminum garnet lasers, yttrium lithium fluoride lasers, excimer lasers, chemical lasers, harmonically oscillated lasers, dye lasers, nitrogen lasers, neodymium lasers, erbium lasers, ruby lasers, titanium-sapphire lasers and diode lasers. Suitable YAG lasers further include frequency doubled and frequency tripled YAG lasers. The wavelength of many YAG lasers can be converted from infrared to the green or UV part of the spectrum, by shining them through special crystals. Because these are conversions from the original infrared wavelength to the second or third harmonic of the fundamental frequency, suitable additional ranges of laser light wavelengths are provided.

A laser source provides a beam with a characteristic power, which depends on the wavelength of the laser light radiation. The laser beam also has a diameter and a surface area of contact. For example, if the diameter of the beam is 1.17 cm, the illuminated surface area will be one square centimeter, since the area is determined by the equation πr² where r is the radius of the beam. If a laser source provides a laser beam having a power of 1 watt and a diameter of 1.17 cm, the beam has an irradiance or intensity of 1 watt per square cm, since the intensity is determined by the equation I=P/A, where P is equal to Power (in watts) divided by A, the area illuminated by the beam in square centimeters. Therefore, if a beam having a power of 1 watt had a diameter of 0.56 cm, the irradiance would be 4 watts/square cm, since the surface area illuminated would be 0.25 cm². If this same laser beam was focused with a focus lens to a smaller diameter and surface area, the intensity would be greatly increased. For example, if the same beam (having a power of 1 watt) were focused to a diameter of 15.7×10⁻¹¹ square cm, the intensity would be 1 watt/15.7×10⁻¹¹ square cm, or 6.37 million watts/square cm. From these calculations, it can be seen that laser beams having relatively low laser power are capable of producing high intensities when focused. For that reason, many laser delivery systems include focus lens(es) to adjust the size and intensity of laser beams.

For the present apparatus, the laser source and laser delivery system should be selected and operated in a manner that avoids, reduces or minimizes damage to the cornea. The wavelength of the laser light can be a wavelength that is generally not absorbed by the cornea. Wavelengths of about 400 nm and longer, alternatively about 632 nm and longer, are preferred. Wavelengths of about 1400 nm and shorter are also preferred. The laser delivery system can include a focus lens that focuses the laser beam at a desired point of ablation rather than at another part of the eye.

However, the present apparatus and methods are not limited to use of lasers for ablation. High intensity focused ultrasound (“HIFU”) is a medical procedure that uses high-intensity focused ultrasound to heat and destroy pathogenic tissue rapidly. It has been used with computerized magnetic resonance imaging (“MRI”), where MRI is employed to identify tumors or fibroids in the body, which can then be destroyed by the ultrasound. In HIFU therapy, ultrasound beams are focused on diseased tissue, and due to the significant energy deposition at the focus, temperature within the tissue rises to 65° to 85° C., thereby destroying or ablating the tissue. The ultrasound beam can be applied to a precisely defined portion of a targeted tissue, and the tissue is ablated by moving or pointing the component that delivers the ultrasound beam to multiple locations. This technique can achieve precise ablation of diseased or other tissue. It is contemplated that HIFU could be used to ablate transitional zone cells to prevent or delay presbyopia, and that HIFU devices could be adapted, to ablate cells in the transitional zone of the crystalline lens.

High intensity focused ultrasound can be employed to apply energy to an eye tissue in order to heat the tissue to a particular temperature while greatly minimizing the collateral damage to adjacent tissues. The ultrasound beams are precisely located, so that the target tissue can be ablated without damaging other parts of the eye. A HIFU transducer can be used to apply focused ultrasonic energy so as to ablate the target tissue without damaging overlying or adjacent tissues. Suitable HIFU transducers include piezoelectric crystals that can focus ultrasonic energy on discrete regions within the eye. Additional discussion and details regarding the use of high intensity focused ultrasound can be found in U.S. Pat. No. 6,128,522; U.S. Pat. No. 6,936,046; U.S. Patent App. Pub. No. 20040039312 and U.S. Patent App. Pub. No. 20080015436.

Radio frequency (RF) energy has been discussed in a method of ocular refractive surgery which employs heat application to reshape the central cornea of a subject. See U.S. Pat. No. 5,423,815. U.S. Pat. No. 6,036,688 discusses an apparatus for performing refractive keratectomy on the eye of a subject by the use of radio frequency energy from a radio frequency generator. It is contemplated by the present inventors that RF energy could be used, and that RF devices could be adapted, to ablate cells in the transitional zone of the crystalline lens.

All materials have a damage threshold, which is a level at which the intensity of the laser beam will cause the material to begin to vaporize or burn. The threshold is the level where damage begins to occur. For materials at room temperature, the damage threshold is dependent upon the intensity of the laser light, how long the light is illuminating the area, and the amount of laser light absorbed by the material.

### Example 1

This example shows that a mechanical or surgical approach can be used to ablate cells in the transitional zone of the crystalline lens. The test subjects for this example are sets of guinea pigs at different ages (for example, one-month-old, one-year-old). Ablation points are made in the transitional zones of one or both eyes of one or more sets of guinea pigs.

The growth rates of the guinea pig crystalline lens are well known and are illustrated below.

Lens structure and function are analyzed at 1 month, three months, and 12 months postsurgery. Structure and function can be analyzed by techniques described in Kuszak et al., “Fibre cell organization in crystalline lenses”, Experimental Eye Research, 78: 3, 673-687 (2004). The equatorial size and lens thickness (between anterior and posterior surfaces) are analyzed and compared to the normal growth rates of the crystalline lens for each age group.

| | | | Change in | Lens | |
|---|---|---|---|---|---|
| Age of Guinea | | Equatorial | Equatorial | Thickness | |
| Pig | | Size (mm) | size | (A/P in mm) | Change in A/P |
| Newborn | | 4.08 +/− 0.05 | NA | 2.59 +/− 0.07 | NA |
| 1 | month | 4.59 +/− 0.06 | 12.5% | 3.58 +/− 0.09 | 38.2% |
| 3 | months | 5.43 +/− 0.04 | 18.3% | 4.42 +/− 0.05 | 23.5% |
| 6 | months | 5.58 +/− 0.04 | 2.8% | 4.49 +/− 0.09 | 1.6% |
| 12 | months | 5.69 +/− 0.09 | 2.0% | 4.50 +/− 0.12 | 0.2% |
| 24 | months | 5.86 +/− 0.07 | 3.0% | 4.66 +/− 0.09 | 3.6% |

Electron microscopy allows the identification of changes in the appearance of the transitional zone. A decrease in the number of cells in the transitional zones, and/or a decrease in the number of fibers cells, allows for greater accommodative potential.

### Example 2

In this example, the procedures used in Example 1 are repeated in different sets of animals (for example, mice or non-human primates).

### Example 3

In this example, sets of guinea pigs of different ages (I month and 1 year old) are selected, and their eyes are maximally dilated. A biomarker is then topically administered to the eyes of the subjects. This biomarker specifically targets cells having nuclei or other organelles exclusively or predominantly found in cells in the transitional zone, so that cells in the transitional zone can be distinguished from cells in the germinative zone and/or from fibers cells. A laser application will be administered to the transitional zone cells of the crystalline lens of the eyes. A specific number (20% and 40%) of cells are ablated using an appropriate energy level, wavelength, pulse width and pulse duration. After ablation the lenses are analyzed by electron microscopy for structural changes and physical measurements of dimension and weight. Lens structure and function are analyzed at 1 month, three months, and 12 months post-surgery. The equatorial size and thickness of the crystalline lens are measured.

In the present specification, use of the singular includes the plural except where specifically indicated. Whenever the term “about” appears before a value, it should be understood that the specification is also providing a description of that value apart from the term “about”, and vice versa.

In the present specification, any of the functions recited herein may be performed by one or more means for performing such functions. With respect to the processes described in the specification, it is intended that the specification also provides a description of the apparatus for performing those processes. With respect to the apparatus described in the specification, it is intended that the specification also provides a description of the components, parts, portions, of such apparatus.

All of the references cited herein, including patents, patent applications, publications, journal articles, standards and any other references, are hereby incorporated in their entireties by reference.

While particular elements, embodiments and applications of the present invention have been shown and described, it will be understood, of course, that the invention is not limited thereto since modifications can be made by those skilled in the art without departing from the scope of the present disclosure, particularly in light of the foregoing teachings.

Although the dependent claims have single dependencies in accordance with U.S. patent practice, each of the features in any of the dependent claims can be combined with each of the features of other dependent claims or the main claim.

We claim:

1. A process for preventing or delaying presbyopia in a subject, wherein the subject has a crystalline lens having a transitional zone, the process comprising the step of ablating cells in a transitional zone of a crystalline lens.

2. The process of claim 1, wherein the process comprises making one or more ablation points are made in the transitional zone of the crystalline lens.

3. The process of claim 1, wherein the crystalline lens further comprises a germinative zone and a pregerminative zone, and the process further comprises distinguishing between cells in the transitional zone and cells in the germinative and pregerminative zones.

4. The process of claim 1, wherein the crystalline lens further comprises a germinative zone and a pregerminative zone, and the process excludes ablating cells in the germinative or pregerminative zones of the crystalline lens.

5. The process of claim 1, wherein the crystalline lens further comprises fiber cells, and the process further comprises distinguishing between cells in the transitional zone and fiber cells.

6. The process of claim 1, wherein the crystalline lens further comprises fiber cells, and the process excludes ablating fiber cells.

7. The process of claim 1, wherein the crystalline lens has a circumference, and the process comprises making ablation points symmetrically around essentially the entire circumference.

8. The process of claim 1, wherein at least about 10% of the cells in the transitional zone of the crystalline lens are ablated.

9. The process of claim 1, wherein the cells in the transitional zone are ablated without forming a cataract or an astigmatic condition.

10. A process for preventing or delaying one or more symptoms of presbyopia in a subject, the process comprising the steps of: selecting a subject prior to detecting a symptom of presbyopia in the subject, and ablating cells in the transitional zone of the crystalline lens.

11. The process of claim 10, wherein the subject is selected based on an increased risk factor for presbyopia.

12. The process of claim 10, wherein the subject is selected based on age.

13. The process of claim 10, wherein the cells in the transitional zone are symmetrically ablated.

14. The process of claim 10, wherein at least about 10% of the cells in the transitional zone of the crystalline lens are ablated.

15. The process of claim 10, further comprising the step of administering to the crystalline lens a label that identifies the transitional cells.
